(19) United States
(12) Patent Application Publication
(10) Pub. No.: US 2005/0255581 A1
(43) Pub. Date: Nov. 17, 2005

(54) Self-organized nanopore arrays with controlled symmetry and order

(75) Inventors: Hong Koo Kim, Pittsburgh, PA (US); Zhijun Sun, Pittsburgh, PA (US)

Correspondence Address: FOLEY AND LARDNER;SUITE 500, 3000 K STREET NW, WASHINGTON, DC, 20007, US

(73) Assignee: UNIVERSITY OF PITTSBURGH

(21) Appl. No.: 10/525,706

(22) PCT Filed: Aug. 22, 2003
(86) PCT No.: PCT/US03/26322

§ 371 Date: Jun. 22, 2005

Related U.S. Application Data
(60) Provisional application No. 60/407,195, filed on Aug. 28, 2002.

(51) Int. Cl. H01L 21/00; C12M 1/34

(52) U.S. Cl. 435/287.2; 438/1

(57) ABSTRACT

An ordered, single domain nanopore array having a macroscale area in a first material is provided. A method of making a nanopore arrays with a controlled pattern include providing a substrate comprising a first surface having a first patter, depositing a first material capable of forming nanopores onto said first-surface having the first pattern, and anodically oxidizing said first material to form the nanopore array with the controlled pattern in the anodically oxidized first material.

The present application claims benefit of U.S. provisional application 60/407,195, filed Aug. 28, 2002 which is incorporated herein by reference in its entirety.

### BACKGROUND OF THE INVENTION

1. Field of the Invention

The present invention relates to methods for arranging highly-ordered nanopore arrays with controlled symmetry onto a foreign substrate surface.

2. Description of the Background

A review of anodic oxidation of compounds to form nanoscale pores may be found in O'Sullivan and Wood, Proc. Roy. Soc. Lon. 317: 511-543. For example, electrochemical (also known as anodic) oxidation of elemental aluminum results in anodic alumina which is a nanoporous material.

Aluminum foils are often used as both a source of elemental aluminum as well as a structure on which the alumina nanopores are formed. Alternatively, aluminum films may be deposited onto a substrate that would then structurally support the subsequent formation of alumina nanopores from the aluminum film. However, the seeding of the alumina nanopores occurs randomly across the face of the aluminum foil or film. Accordingly, the domain size (i.e. an area of nanopores that has the same triangular symmetry) that is generated on bulk aluminum foil is usually limited to a micrometer scale, thus reducing the utility of these materials for applications that require larger areas of uniform symmetry.

### SUMMARY

A preferred aspect of the present invention provides an ordered, single domain nanopore array having a macroscale area in a first material.

Another preferred aspect of the present invention provides a device comprising a nanopore array having an ordered predetermined pattern of nanopores in a first layer of the device.

Another preferred aspect of the present invention provides a method of making a nanopore array with a controlled first pattern. The method includes providing a substrate comprising a first surface having a first pattern, depositing a first material capable of forming nanopores onto said first surface having the first pattern, and anodically oxidizing said first material to form the nanopore array with the controlled first pattern in the anodically oxidized first material.

### BRIEF DESCRIPTION OF THE DRAWINGS

In the following figures, like reference characters designate the same or similar elements, which figures are incorporated into and constitute a part of the specification.

FIG. 1A is a schematic top view of an apparatus for performing holographic lithography.

FIGS. 1B and 1C are schematic side cross sectional views of a method of making a photoresist pattern according to the preferred embodiments of the present invention.

FIG. 2A is a scanning electron micrograph of the cross-section of a 1D grating patterned photoresist layer on a substrate.

FIGS. 2B and 2C are scanning electron micrographs of square and triangular symmetry photoresist grating patterns, respectively, on a silica substrate.

FIG. 3A is a schematic three dimensional view of steps in methods of making an array according to the preferred embodiments of the present invention.

FIG. 3B is a scanning electron micrograph of a two dimensional square pattern in a chromium hardmask layer.

FIG. 3C is a scanning electron micrograph of an anodic alumina nanopore array formed on a silica substrate.

FIGS. 4A, 4B and 4C are schematic side cross sectional views of steps in methods of making an array according to an alternative preferred embodiment of the present invention.

FIG. 4D is a scanning electron micrograph of an aluminum film with thickness of approximately 350-400 nanometers on a 1-D grating.

FIG. 4E is a scanning electron micrograph of a nanopore array according to a preferred embodiment of the present invention.

FIG. 4F is a scanning electron micrograph of a prior art nanoporous alumina film.

FIG. 5A is a scanning electron micrograph of square-lattice arrangement of square-shaped pores with square arrangement of pores observed across the entire grating area.

FIG. 5B shows a higher magnification of a square-lattice arrangement of square-shaped pores.

FIG. 5C depicts a cross-sectional image of alumina nanopores showing that the pores grow well aligned to the center of the corrugation bottoms.

FIG. 5D is a scanning electron micrograph of alumina pores obtained from a triangular-lattice 2D-grating-patterned aluminum film that was deposited onto a silica substrate at low and high (inset) resolution.

FIG. 5E is a top view of nanopore arrays according to the preferred embodiments of the present invention.

FIG. 6A is a schematic side cross sectional view of an array according to the preferred embodiments of the present invention.

FIG. 6B is a schematic side cross sectional view of an electroplating bath used to make the array of FIG. 6A.

FIGS. 7A, 7B, 7C and 7D are schematic side cross sectional views of a method of making an array according to the preferred embodiments of the present invention.

FIG. 8 is a schematic three dimensional view of a device according to a preferred embodiment of the present invention.

The nanopore array may be formed in a metal oxide material formed by anodic oxidation, such as an anodically oxidized aluminum oxide. Alternatively, the nanopore array may be formed in any other suitable substrate material, such as a semiconductor (i.e., silicon, SiGe, SiC, III-V or II-VI material), glass, ceramic, metal or other material by etching nanopores in the substrate material using the metal oxide film containing the nanopores as a mask and then optionally removing the metal oxide film.

Preferably, the metal film on the substrate comprises a thin metal film rather than a bulk metal foil. However, a bulk metal foil may also be used by photolithographically forming the depressions in a surface of the metal foil and then anodically oxidizing the foil to selectively form the nanopore array.

The term “nanopore” as used herein, is a groove having a diameter of 500 nm or less. Preferably, but not necessarily, a nanopore has a diameter of less than 100 nm, such as about 5-10 nm. Preferably, an unetched nanopore does not extend all the way through the thickness of material that it is in. However, a nanopore depth may be extended by further etching. The term “domain” as used herein means a region containing repeating, similarly shaped units of nanopores, such as linear or polygonal units of nanopores, for example, where the nanopores are aligned in a straight or curved line or comprise vertices of a polygon. The term “ordered” as used herein means a non-random arrangement. An “ordered domain” is a region having a non-random arrangement of repeating units of nanopores. The term “symmetric” as used herein means having a correspondence of form and arrangement of parts on opposite sides of an imaginary boundary line between smallest repeating units of nanopores. The term “predetermined” as used herein means preselected, as in nanopores located in preselected rather than in random locations. The term “film” as used herein means a thin film deposited by thin film deposition methods, such as a film having a thickness less than 10 microns, preferably less than 1 micron. The term “macroscale area” as used herein comprises a macroscopic area that is visible to the naked eye, such as, for example, an area of at least one centimeter, preferably 1 to 100 centimeters.

Preferably, the nanopore array is substantially defect free in the single domain. In other words, the single domain contains no or almost no nanopores which are randomly arranged outside the repeating units of nanopores. Most preferably, as will be described in more detail below, the single domain nanopore array comprises nanopores arranged in a predetermined ordered symmetric pattern, with the nanopores located at each vertex of the polygon. For example, the nanopores may be arranged in an ordered square or triangular symmetric pattern. Alternatively, the single domain nanopore array comprises nanopores arranged in a one dimensional grating pattern where the nanopores are aligned in order along a grating vector direction but are not aligned along a grating line direction.

In a preferred embodiment of the present invention, a pattern is formed over macro-scale areas of a substrate to promote the self aligned formation of ordered arrays of nanopores over large areas of the substrate. The nanopore arrays provide systems and methods for the production of self-ordered nanostructures on wafers with controlled symmetry and order. The regular arrangement of nanopores also allows for the small scale manipulation of substrate. Accordingly, numerous nanoscale electronic, photonic, and chemical devices may be designed, engineered, and constructed, such as nanocircuitry and nanomachines that may be produced from such ordered arrays on a substrate.

A method of making a nanopore array with a controlled pattern includes providing a substrate having a first surface having a first pattern, depositing) a first material onto the first surface of the substrate, and anodically oxidizing the first material to form the nanopore array with the controlled pattern in the anodically oxidized first material.

In a preferred embodiment, the substrate is a silicon or glass (i.e., silica (SiO₂) or other glass) and the first material is aluminum, which is anodized to form a nanoporous anodic alumina. However, one of skill in the art will recognize that the methods and compositions described herein are applicable to a wide variety of substrates including, but not limited to, other semiconductor substrates, such as gallium arsenide, indium phosphide, gallium phosphide, gallium nitride, and silicon carbide, as well as plastic substrates, ceramic substrates, such as sapphire, quartz substrates and metal substrates. The substrates may comprise a single layer, such as an uncovered silicon wafer, or a plurality of layers, with the pattern being formed in the upper layer. In addition, the nanopore array may be formed in any suitable material that may be oxidized, such as by anodic oxidation, to form a nanopore array. For example, instead of aluminum, other metals which form an anodic oxide, such as titanium (which forms a titanium oxide (Gong et al., (2001) J. Mat. Res., vol. 16(12), pp. 3331-3334) upon anodic oxidation), tantalum (which forms Ta₂O₅ upon anodic oxidation), niobium or alloys thereof may be used. In general, any metals or semiconductors that can be oxidized to form nanoporous structures may be used. Furthermore, as will be described below, the anodically oxidized material may be used as a temporary or sacrificial template mask to transfer the nanopore array to the substrate and which is then removed. Thus, the nanopore array may be located in any solid material.

The pattern in the substrate may be formed by any suitable method. Preferably, the pattern is formed by photolithographic patterning and etching. The photolithographic patterning includes forming a photoresist layer on the first surface of the substrate, such as the upper surface of the substrate, selectively exposing the photoresist layer and patterning the exposed photoresist layer to form a patterned photoresist layer.

Preferably, holographic lithography is used to expose a pattern in the photoresist layer. The exposed photoresist layer is then patterned to form a photoresist pattern of ridges or corrugation across the surface of the substrate.

The etching step includes etching the first surface of the substrate using the photoresist pattern as a mask to form the first pattern in the first surface of the substrate. Preferably, but not necessarily, the photoresist pattern is removed after etching the substrate.

A material, such as aluminum, is then deposited onto the patterned surface of the substrate, such as a ridged or corrugated surface. The thickness of the material is sufficiently thin to allow the pattern of the upper surface of the substrate to be maintained in the upper surface of the material across the surface of the substrate. For example, upon anodic oxidation, aluminum is converted to alumina with an array of nanopores. Since the deposited material, such as aluminum, assumes the shape or pattern of the underlying substrate, the nanopores form in the crevices, recesses or troughs in the upper surface of the anodized material. Since the protrusions and recesses in the substrate surface are arranged in a predetermined pattern, a regular pattern or a regular symmetric pattern, the nanopores are arranged in a predetermined, regular and/or symmetric pattern in the anodized material across the face of the substrate. Thus, this method provides for the rapid and efficient production of symmetrical arrays of nanoporous structures across large areas of substrate.

Generally, the arrangement of the nanopores (i.e., both the order and symmetry of nanopore arrays) can be well controlled and guided by nanoscale surface patterns, such as corrugation patterns in an aluminum film on the substrate. The preferred embodiment of the present invention provides for a holographic lithography technique to be utilized in conjunction with a conformal film deposition process to generate patterns that form a lattice across a macroscale area of the aluminum film prior to anodization. The lattice may be in predetermined arbitrary shapes, with square and triangular lattices being two presently-preferred embodiments. The lattice provides a structured pattern for seed points for the formation of nanopores. The nanoscale patterning of films thereby allows for the formation of highly ordered (defect-free, single-domain) pore arrays on a macroscale area.

A method of making a nanopore array according to a first preferred embodiment of the present invention is described below. First, an area of a substrate is initially coated with photoresist. For example, an entire upper surface of the substrate is covered with a photoresist layer. The coating of substrate with photoresist may occur by dipping, spraying, spin coating, or any other suitable procedure than produces a smooth layer of photoresist with controllable and uniform thickness across the area of desired dimension. For example, the photoresist may be coated as a 100-150 nanometer layer onto a silica substrate.

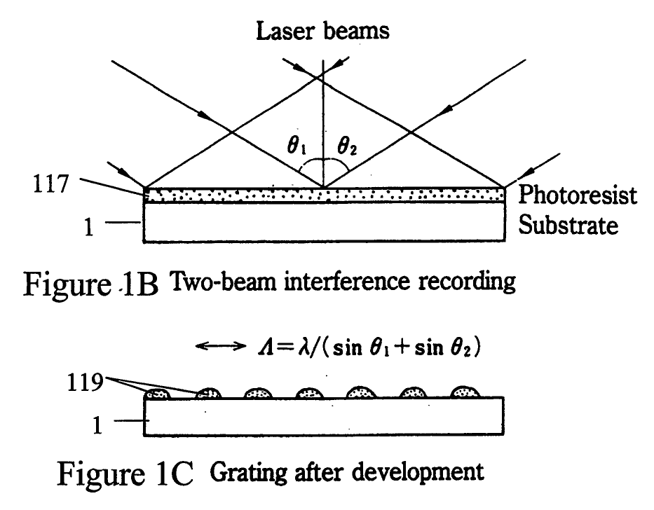

The photoresist layer is then patterned using holographic lithography, as illustrated in FIG. 1A. Of course, if desired, any other suitable method may be used to pattern the photoresist layer, such as non-holographic lithography or selective electron beam exposure.

An exemplary holographic lithography system is illustrated in FIG. 1A. The system includes a vibration free optical table or bench 100 supporting a radiation source, such as a laser 101, an optional shutter 103, an optional first mirror 105, a beam splitter 107, secondary mirrors 109, various beam shaping optics, such as filters 111 and lenses 113, and a sample holder 115, such as a rotatable stage.

In a presently-preferred embodiment, the laser 101 is a helium-cadmium laser (325 nanometer wavelength, 15 milliwatt output power), which emits a beam that is expanded and collimated into a beam diameter of one to two centimeters and then split into two equal intensity beams by the splitter 107. The two beams are then preferably recombined onto a photoresist layer 117 (for example, SHIPLEY 1805 positive photoresist diluted with thinner P solution in 1:1 volume ratio and having a thickness of about 300 to 400 nm) located over a substrate 1, such as a silica substrate, to form the interference pattern, as shown in FIG. 1B.

Where the two beams converge, an interference pattern comprised of multiple parallel lines of intense light is generated. The parallel lines of intense light occur with a particular periodicity which may be adjusted by changing the incident beam angle. Further adjustment of the periodicity may be accomplished by changes in optics, e.g., changes in the wavelength of the light source, and/or the refractive index of the ambient dielectric adjacent to the photoresist. Thus, the photoresist is exposed where the two beams converge and not exposed where the two beams do not converge. The length, A, shown in FIG. 1C is equal to the peak wavelength of the split laser beams divided by (sin θ₁+sin θ₂), where θ₁ and θ₂ are the angles of the laser beams with the normal to the photoresist surface, as shown in FIG. 1B.

The selective exposure leaves the photoresist layer 117 with exposed and non-exposed regions. The holographic exposure is preferred because it forms slit shaped exposed and non-exposed regions in the photoresist layer which can then be used to form slit shaped ridges and grooves in the substrate. The exposed photoresist layer is then patterned, as shown in FIG. 1C. If the photoresist layer 117 is a positive photoresist layer, then the exposed regions are removed by a suitable solvent, while leaving the unexposed regions as a photoresist pattern 119 on the substrate 1, as shown in FIG. 1C. If the photoresist layer 117 is a negative photoresist layer, then the unexposed regions are removed by a suitable solvent, while leaving the exposed regions as a photoresist pattern 119 on the substrate 1.

The grating pattern size is scalable to larger values with appropriate changes in the optics. The grating line pattern is preferably referred to as a one-dimensional or 1D pattern. Preferably the exposure intensity and exposure time are adjusted such that the substrate surface is fully revealed for approximately half of the grating period.

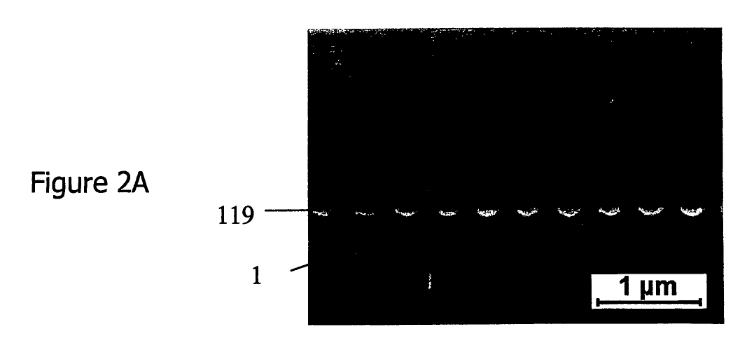

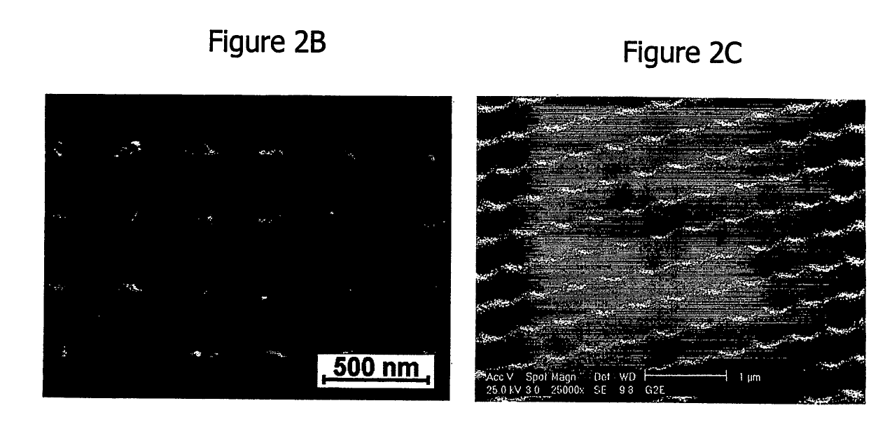

FIG. 2A illustrates a scanning electron micrograph of the cross-section of a 1D grating patterned photoresist layer on a substrate. The corrugation depth of the photoresist grating in this example is approximately 120 nanometers.

In a presently-preferred embodiment, the photoresist-coated substrate is double or triple exposed to the incident laser light with 60 or 90 degrees of rotation between the exposures for triangular or square symmetry, respectively. Preferably, the substrate is rotated between each exposure, while the laser beams remain unmoved. However, if desired, the laser beams may be relatively rotated between exposures using rotational optics while the substrate remains unmoved. Preferably, electro-optic rather than mechanical beam rotational devices are used to rotate the laser beams between exposures.

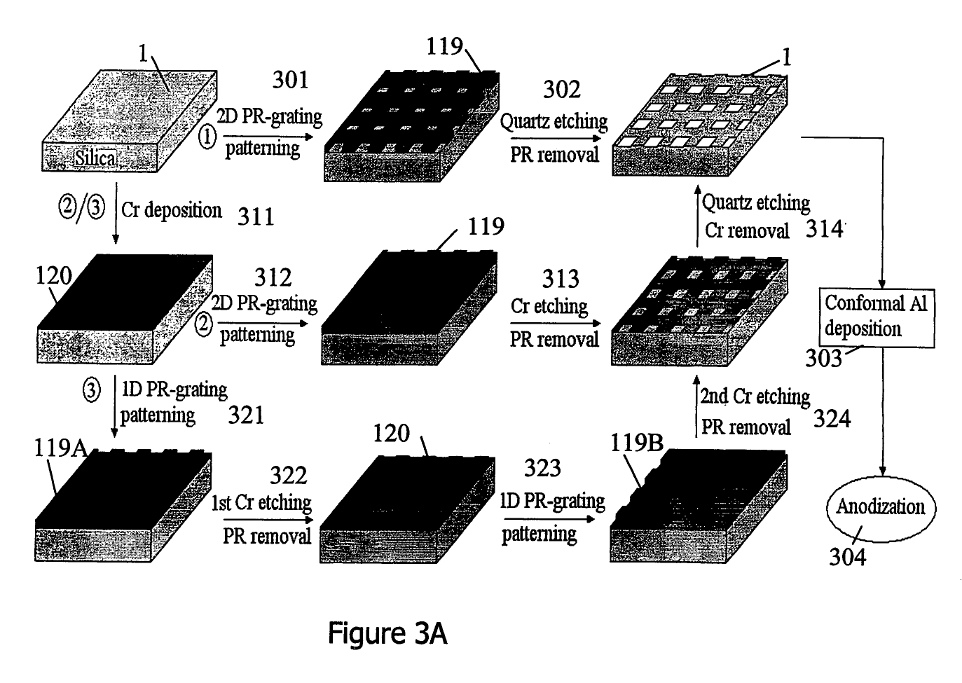

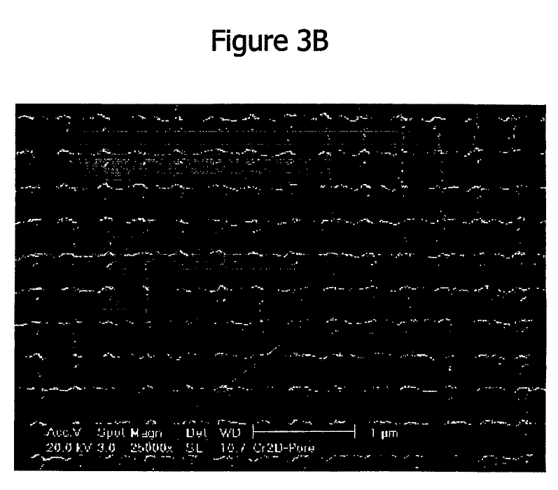

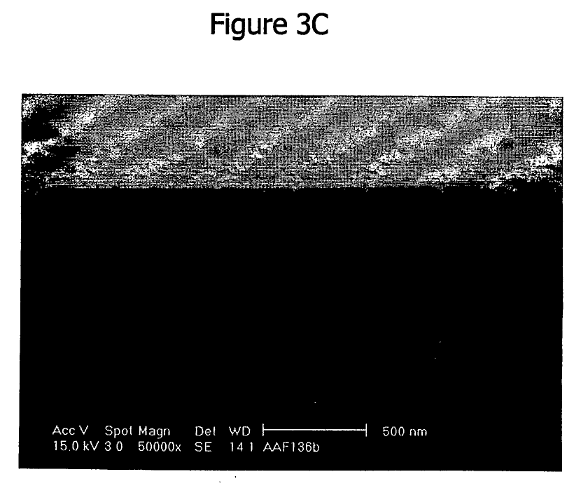

FIG. 2B illustrates a presently-preferred embodiment wherein a square symmetry photoresist grating pattern is developed on a silica substrate. Thus, the etched photoresist pattern displayed is essentially the surface using the photoresist as an etch mask. For example, as shown in FIG. 3A, a substrate 1, such as an about 600 micron thick silica substrate is provided. In the first preferred patterning method, in step 301, a photoresist layer is patterned into a two dimensional pattern 119, by any suitable method described above. For example, in the photoresist pattern 119, the crossed regions may have a thickness of about 80 nm and the ridge regions may have a thickness of about 40 nm. Then, in step 302, the substrate 1 is etched using the patterned photoresist 119 as a mask to transfer the pattern to the substrate. For example, the corrugation depth in the substrate may be about 10 to about 20 nm. The photoresist is then removed from the substrate.

In a second preferred method, a two dimensional photoresist pattern is transferred to a hardmask layer using an etching process, and then the patterned hardmask layer is used a hardmask in etching a substrate surface. For example, in the second preferred patterning method, in step 311, a hardmask layer 120 is deposited on the substrate 1. The hardmask layer may comprise any suitable hardmask material, such as an about 10 nm thick Cr layer or another other suitable metal layer. In step 312, a photoresist layer is patterned into a two dimensional pattern 119 over the hard mask layer 120, by any suitable method described above. For example, the crossed regions in the photoresist pattern 119 may have a thickness of about 80 nm and the ridge regions may have a thickness of about 40 nm. Then, in step 313, the hardmask layer is etched using the patterned photoresist 119 as a mask to transfer the pattern to the hardmask layer 120. The photoresist is then removed from the patterned hardmask layer, if desired. In step 314, the substrate 1 is then etched using the patterned hardmask layer (and the photoresist if not removed previously) as a mask. For example, the corrugation depth in the substrate may be about 10 to about 30 nm, preferably about 20 to about 30 nm.

In a third preferred method, a first one dimensional photoresist pattern, having its grating lines aligned in a first direction, is transferred to a hardmask layer. Then, the process is repeated with a second one dimensional photoresist pattern having its grating lines aligned in a second direction different from the first direction. The patterned hardmask layer is then used as a hard mask in etching a substrate surface. For example, in the third preferred patterning method, in step 311, a hardmask layer 120 is deposited on the substrate 1. The hardmask layer may comprise any suitable hardmask material, such as an about 50 nm thick Cr layer or another other suitable metal layer. Then, in step 321 a first photoresist layer is patterned into a one dimensional pattern 119A with its grating lines extending in a first direction over the hard mask layer 120, by any suitable method described above. The photoresist grating thickness may be about 80 nm. Then, in step 322, the hardmask layer is etched using the patterned photoresist 119A as a mask to transfer the pattern to the hardmask layer 120. For example, the hard mask layer may be etched part of the way (such as half way) through its thickness, such as to about 25 nm, during this step. The photoresist 119A is then removed from the patterned hardmask layer. In step 323, a second photoresist layer is patterned into a one dimensional pattern 119B with its grating lines extending in a different second direction, over the patterned hard mask layer 120, by any suitable method described above. A square lattice pattern is formed if the grating line directions are perpendicular and a triangular lattice pattern is formed if the grating line directions differ by 60 degrees. Then, in step 324, the patterned hardmask layer 120, is etched again using the patterned photoresist 119B as a mask to transfer the pattern to the hardmask layer 120. Preferably, the hardmask layer is again etch part of the way, such as half way, so that the hardmask layer thickness is about 50 nm in the crossed regions, about 25 nm in the ridge regions and zero nm between the ridge regions (i.e., an opening is formed between the ridge regions). In step 314, the substrate 1 is then etched using the patterned hardmask layer as a mask to form a two dimensional pattern in the substrate. The second photoresist 119B is removed from the patterned hardmask layer before or after patterning of the substrate. For example, the corrugation depth in the substrate may be about 10 to about 50 nm, preferably about 30 to about 50 nm.

It should be noted that two dimensional patterns other than the illustrated square patterns may be formed in the substrate. In the second and third methods, the patterned hardmask layer may be removed from the substrate prior to the deposition of the anodizable metal film or the anodizable metal film may be deposited directly onto the patterned hardmask layer.

The second substrate patterning method potentially offers an advantage over the first method in that it allows deeper etching of substrate with the use of a hardmask layer. The third method potentially offers an advantage over the second method in that each hardmask layer line remains well connected after etching and this helps develop well-defined (isolated) openings in the hardmask. FIG. 3B is a micrograph of a two dimensional square pattern in a chromium hardmask layer formed on a silica substrate by the third patterning method described above.

Table I below provides the preferred, exemplary plasma etching conditions that may be used in the pattern transfer processes described above.

TABLE I

| | Cr | SiO₂ (silica) |
|---|---|---|
| Gas | Cl₂ + O₂ | CF₄ + O₂ |
| Flow rate (sccm) | 24 + 6 | 36 + 4 |
| Pressure (mTorr) | 10 | 15 |
| RIE power (W) | 10 | 50 |
| ICP power (W) | 100 | 75 |
| Etch rate ratio | PR:Cr = 3:4 | Cr:SiO₂ = 1:12 |

In the next step of the process, the material that is to form the nanoporous structures, such as the anodizable metal film, is preferably deposited directly onto the patterned substrate and/or onto the hardmask, if a hardmask is present. Deposition may occur by any suitable deposition method, such as vacuum evaporation, such as thermal or electron beam evaporation, MOCVD, MBE, sputtering, electroplating or electroless plating. Preferably, the metal film is evaporated in a high vacuum (typically 10⁻⁶ Torr or lower pressure) system, so that the mean-free-collision-path of evaporated particles is larger than the distance from the source to the substrate. These conditions would result in a deposition of evaporated material on substrate, producing conformal profiles of films deposited on a patterned surface. Thus, the pattern in the upper surface of the substrate is transferred to the upper surface of the metal film.

For example, as shown in step 303 in FIG. 3A, an about 300 nm to about 800 nm thick alumina film is deposited on a patterned substrate and/or on a patterned hardmask layer. This metal layer is then anodized in step 304 to form the nanopore array. FIG. 3C is an SEM micrograph (cross-sectional image) of an anodic alumina nanopore array formed on a silica substrate. The two-step 1D-grating patterning process (i.e., the third method described above) is used in developing the 2D Cr hardmask pattern. The substrate corrugation can be seen near the bottom of pores. An aluminum film with an initial thickness of 350 nm is deposited on the corrugated substrate and is then anodized at 140 V for 40 min.

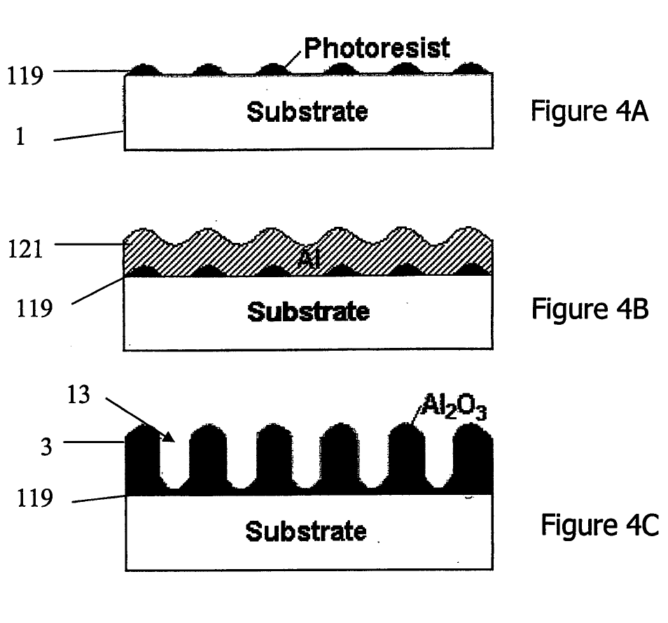

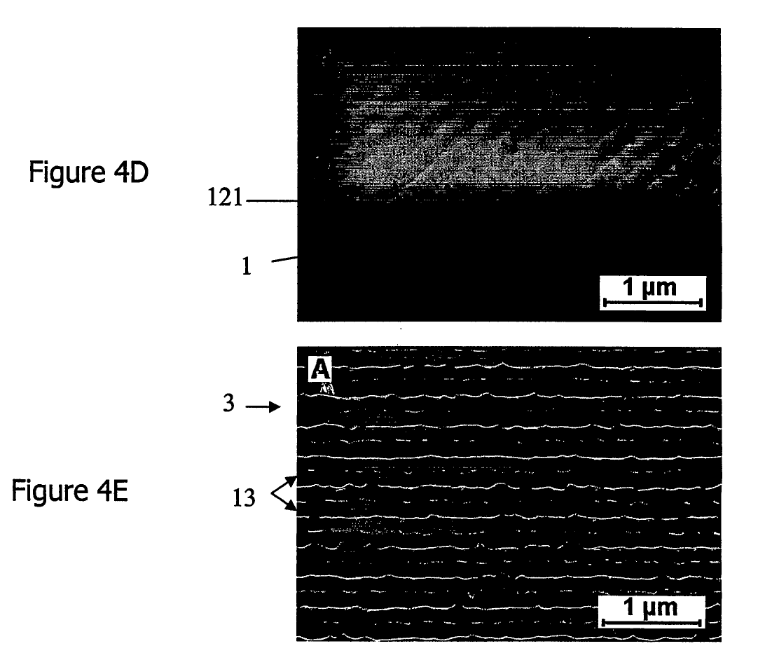

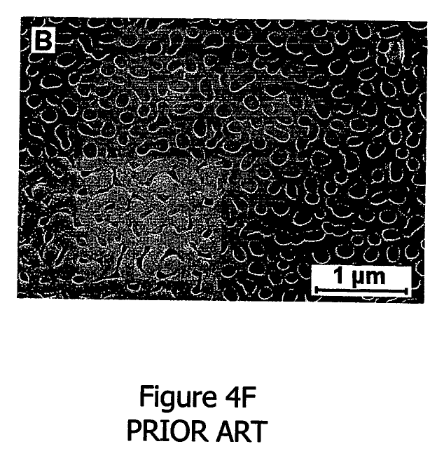

In another alternative preferred embodiment, the metal film may be deposited directly on the photoresist pattern. FIGS. 4A-4C schematically illustrate a process of growing ordered, single-domain, alumina nanopore arrays on a substrate whose surface is corrugated with a photoresist grating pattern. In this case, the substrate is not etched after forming the photoresist pattern and metal layer is deposited directly onto the photoresist pattern. Thus, the photoresist pattern is transferred to the upper surface of the metal film. For example, as shown in FIG. 4A, the photoresist pattern 119 is formed on the substrate 1 by any suitable method described previously. The metal layer, such as an aluminum layer 121, is deposited onto the photoresist pattern 119, as shown in FIG. 4B. Then, as shown in FIG. 4C, the metal layer 121 is anodically oxidized to form the nanopore array 3 containing nanopores 13. It should be noted that the above described aluminum film may be a pure aluminum film or an alloy of aluminum in which aluminum is more than 50 percent by weight, such as an Al—2% Cu alloy.

FIG. 4D illustrates a micrograph of the preferred embodiment in which an aluminum film has been deposited onto a 1-D photoresist pattern. In the preferred embodiment displayed in FIG. 4D, an aluminum film 121 with a thickness of 350-400 nanometers is deposited onto the 1-D photoresist pattern on a substrate using a thermal evaporation method with a 99.999% (5N) purity aluminum source. The deposited film surface preferably conforms to the corrugation profile of the photoresist pattern with nearly the same amount of corrugation depth, that is, approximately 100 nanometers. Preferably, the metal film is less than 1 micron thick, more preferably less than 500 nanometers thick.

Anodic oxidation of the deposited metal film is then carried out. In a presently-preferred embodiment, an aluminum film that has been deposited onto a silica substrate is anodically oxidized in dilute electrolyte (1 H₃PO₄+800 H₂O in volume ratio) at room temperature using a platinum wire as a counter electrode. The anodization is preferably conducted under a constant voltage mode for about 40 minutes. A different anodization duration may be used for different materials and different film thicknesses. The anodic voltage is chosen such that the expected pore distance matches the grating period, for example 140 volts for a 350 nanometer grating period. In a naturally-formed alumina pore array, the interpore distance is proportional to the anodization voltage, i.e. about 2.5 nanometers/volt. The voltage may be varied for anodizing different portions of the metal layer to form pores with a variable period. After anodization, the samples are as-grown without any postanodization etching [the inset to FIG. 4F], and this indicates that pore nucleation was completely random in the unpatterned film case.

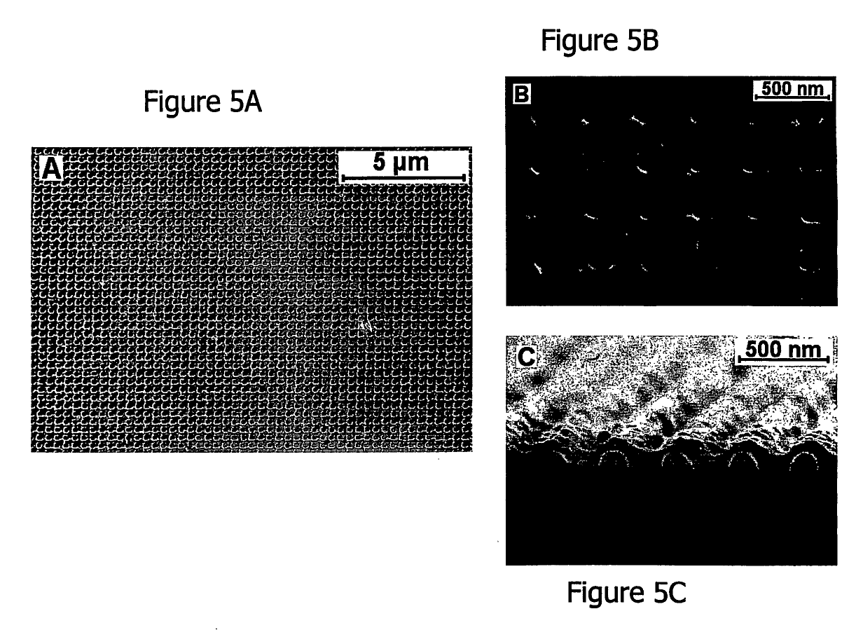

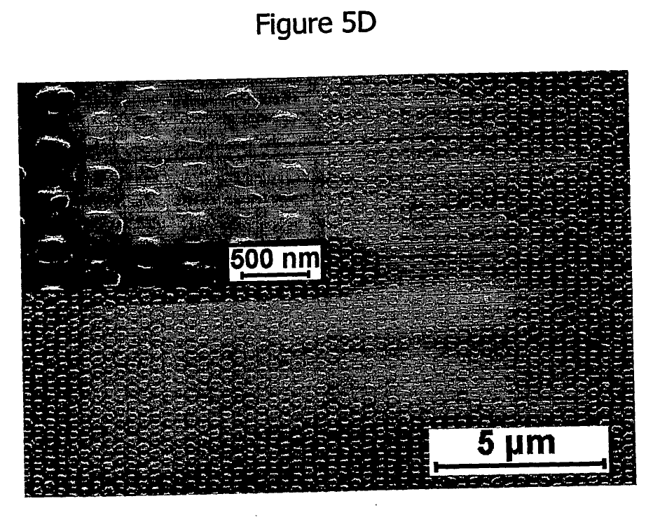

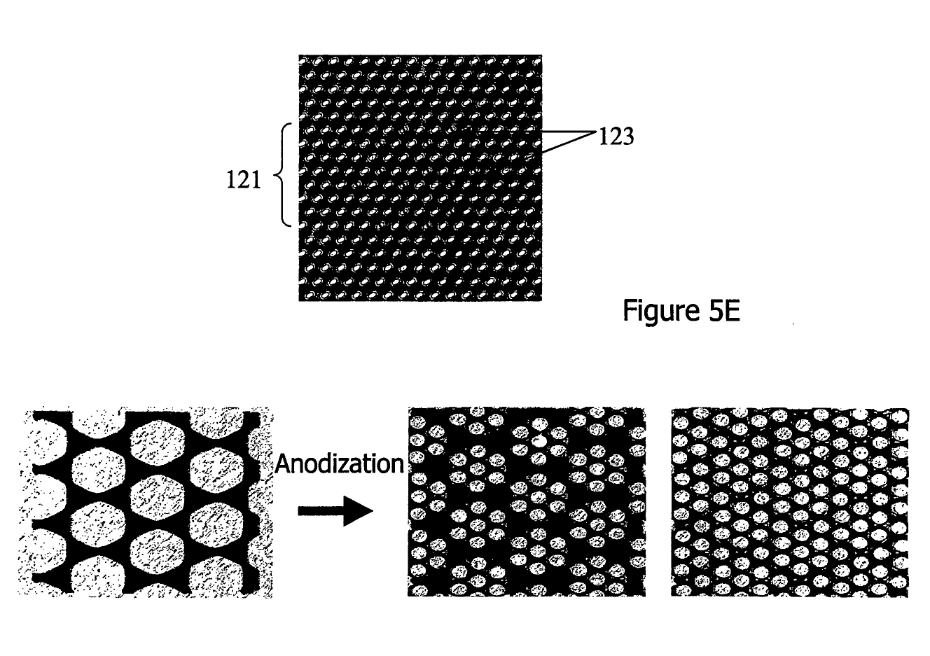

FIG. 5A illustrates a low resolution scanning electron micrograph of a square-lattice arrangement of square-shaped pores with square arrangement that is formed by anodic oxidation of deposited aluminum. The arrangement of pores across the entire surface is extremely regular, corresponding to the etched photoresist pattern. FIG. 5B is a higher resolution image of square-shaped pores. FIG. 5C shows a cross-sectional view of alumina nanopores formed using a 1-D etching pattern. The nanopores show uniform depth of about 400 nm and the pore bottom has a concave, hemispherical shape with barrier layer thickness of about 100 nm. The pores grow well aligned to the center of recess or corrugation bottom region. Thus, nanoscale periodic patterning of the aluminum film surface can compensate the randomizing effect of grain boundaries in aluminum films from the beginning of pore formation, and can control/guide development of order throughout the pore growth process across the entire pattern area.

FIG. 5D illustrates an embodiment of the present invention where the photoresist-coated substrate was exposed to diffraction patterns that were rotated 60 degrees with respect to one another. The resulting triangular arrangement of alumina pores is displayed at both high and low magnification. The single-domain, triangular arrangement of pores is observed across the entire pattern area of at least about one cm².

The elliptical pore shape is considered a reflection of the grating pattern symmetry, similar to the square lattice case discussed above. Each concave bottom is surrounded by four corners, which form a rhombus-shape sublattice with two-fold symmetry. The in-plane radius of curvature at the corners of the major axis is smaller than that at the minor-axis corners. Therefore the electric field (and oxide dissolution) is believed to be the strongest (fastest) along this major-axis direction. This is believed to induce the pores to take an elliptical shape.

FIG. 5E is a schematic illustration of nanopore arrangement guided by nano and micro scale substrate surface patterns. For example, as illustrated in the upper portion of FIG. 5E, a hexagonal supercell 121 contains seven cells 123 each containing seven nanopores. If the nanopore array is anodized at a higher voltage to form macropores, than that used to form the nanopore array, then the single domain nanopore array is separated into a plurality of cells before or after forming the nanopores. Each cell contains nanopores arranged in a predetermined ordered symmetric pattern. In other words, the cells or ridges are separated by macropores, while each cell contains nanopores. Alternatively, the metal film may be patterned into cells by lithography or metal cells may be selectively formed on a substrate pattern and then anodized to form the nanopores in each cell. Such an arrangement is illustrated in the lower portion of FIG. 5E.

The embodiment described above involves substrate patterning prior to film deposition. An alternative process may also be employed to generate surface pattern on deposited metal films. Initially, a metal film, such as an aluminum film, may be deposited onto a patterned or unpatterned substrate. Subsequently, a photoresist layer is formed on the metal film. The photoresist layer is exposed and patterned as described above to form a pattern.

If desired, so-called hard mask layer, such as a silicon oxide, silicon nitride, silicon oxynitride, or other suitable material layer which enhances the adhesion of the metal layer to the photoresist layer, may be formed between the metal film and the photoresist layer. The hard mask layer enhances the maximum etch depth in the pattern-transfer etching process.

The metal film is then wet or dry etched using the photoresist pattern as a mask to transfer the photoresist pattern into the upper surface of the metal film. If the hardmask layer is present, then the hardmask layer is first etched using the photoresist pattern as a mask, and then the metal film is etched using the patterned hardmask layer as a mask. The photoresist layer may be removed before or after etching of the metal film using the hardmask layer as a mask. Preferably, the hardmask layer is removed after patterning the metal film, such that the entire patterned metal film is exposed. The patterned metal film is then anodically anodized using the anodization process described above to form the nanopore array.

The resulting alumina pores made by the methods of the first and second preferred embodiment typically show uniform depth (400 nanometers) and the pore bottom has a concave, hemispherical shape with barrier thickness of approximately 300 nanometers. The pores typically grow well aligned to the center of the corrugation bottom region. Thus, the nanoscale periodic pattern of a metal film, such as an aluminum film, can compensate for the randomizing effect of grain boundaries typically observed in aluminum films.

In another preferred embodiment of the present invention, the nanopore array in the anodically anodized metal oxide is used as a mask to form the nanopore array in the substrate. In this embodiment, the nanopore array is first formed in an anodically oxidized metal oxide film by any suitable method described above. The metal oxide layer is then used as a mask to etch the substrate. Any suitable wet or dry etching medium which preferentially etches the substrate material over the metal oxide material may be used to etch the substrate. Preferably, a dry, anisotropic etching medium is used (i.e., etching gas or plasma). The etching medium permeates through the nanopores and etches the substrate material below the nanopores. Thus, the nanopore pattern is transferred from the metal oxide film to the substrate material. The nanopores may extend to any desired depth in the substrate, depending on the etching medium, etch duration and substrate material(s). If desired, the metal oxide film may be removed after etching the substrate. Alternatively, the metal oxide film may be left on the substrate after etching the substrate and incorporated into a device that includes the substrate containing the nanopore array.

The large areas of the substrate that possess ordered arrays of nanoporous metal oxide film and/or the substrate containing the nanopore array have a variety of industrial applications. These applications include, but are not limited to microelectronics, the construction of optical nanodevices, fuel cells, nano-structuring, and chemical catalyst applications.

Preferably, but not necessarily, the devices include the nanopore array in the metal oxide layer and/or in the substrate, where the nanopores are filled by a material different than the material in which the nanopores are located. If desired, different materials are provided into different nanopores. Thus, different devices may be formed in different regions of the nanopore array to form a multifunctional nanosystem on a chip or substrate. For example, logic and memory devices, or any other suitable combination of devices described below may be formed on the same chip or the same substrate. If desired, different pore geometries may be formed in different domains or areas on the same substrate to facilitate the multifunctional nanosystem.

The nanopores may be filled by any suitable method. For example, one or more material films may be conformally deposited over the nanopore array, such that the material protrudes into the nanopores. If desired, the material may be removed from above the nanopores to leave isolated islands of material located in the nanopores. For example, a film located above the metal oxide film or substrate containing the nanopores, may be removed by chemical mechanical polishing which stops on the metal oxide or substrate material (i.e., which acts as a polish stop). This polishing step leaves isolated material islands located in the nanopores of the array. Other removal methods, such as etchback, may be used to remove the material film overlying the nanopore array.

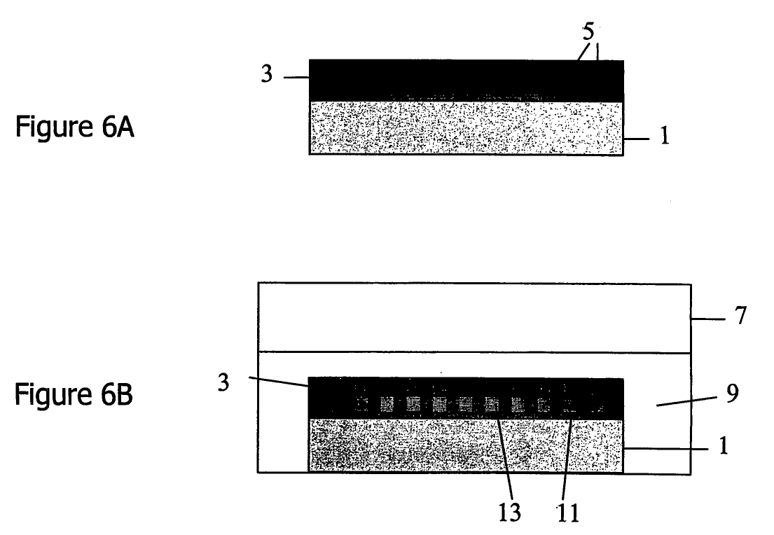

Alternatively, the material is selectively deposited into the nanopores. For example, after forming the nanopore array 3 on a substrate 1, metal islands 5 are selectively grown in the nanopores, as shown in FIG. 6A. One preferred method of selectively growing metal islands inside the nanopores in a metal oxide layer is an electroplating method illustrated in FIG. 6B. In this embodiment, the nanopore array 3 is formed on a conductive or a semiconducting substrate 1. The substrate 1 may comprise a metal layer, such as a metal layer which is not anodically oxidized, or a doped semiconductor layer, such as silicon, gallium arsenide or gallium nitride. The substrate 1 may also comprise a radiation transparent substrate to used in the devices that require light transmission through the substrate. The substrate 1 and array 3 are then provided into an electroplating bath 7 containing a liquid metal 9. A potential difference (i.e., a voltage) is applied between the substrate 1 and the array 3. Since the array 3 is thinner in regions 11 below the nanopores 13, a voltage gradient exists in these regions 11. This causes the metal 9 from bath 7 to selectively deposit into the nanopores 13.

If desired, the electroplating method may be used to selectively fill the nanopores 13 with metal 9 from bath 7. The metal 9 may be any metal which may be deposited into metal oxide pores by electrodeposition, such as Ni, Au, Pt and their alloys.

In an alternative preferred aspect of the present invention, the nanopores 13 are filled only part of the way with the metal 9 during the electroplating step. In this case, the metal 9 may be any metal which can act as a catalyst for selective material vapor deposition. For example, the metal 9 may be Au. The array 3 with the catalyst metal 9 formed on the bottom of the nanopores 13 is then transferred to a vapor deposition chamber, such as a chemical vapor deposition chamber. Islands 5 are then selectively grown on the catalyst metal 9 by selective vapor deposition. The islands 5 may comprise any material which may be selectively deposited on a catalyst metal 9, but not on metal oxide walls of the nanopore array 3. For example, this material may comprise a metal such as Al or Ag.

If the nanopore array 3 is formed on a temporary substrate 1, then the temporary substrate may be removed from the array before or after the formation of the metal islands 5 on the array 3. The temporary substrate may be removed by selective etching, polishing or chemical mechanical polishing of the substrate, by selective etching of a release layer (not shown for clarity) located between the temporary substrate and the array, or by peeling the substrate away from the array. In case of peeling, one or more peel apart layers may be located between the substrate and the array. The peel apart layer(s) have a low adhesion and/or strength such that they can be separated mechanically from each other or from the array and/or the substrate. A permanent device substrate, such as a transparent substrate or another part of the final device, such as a photodetector, is then attached to the array 3 before or after forming the metal islands 5 in the array, on the same side and/or on the opposite side of the array 3 from where the temporary substrate was located.

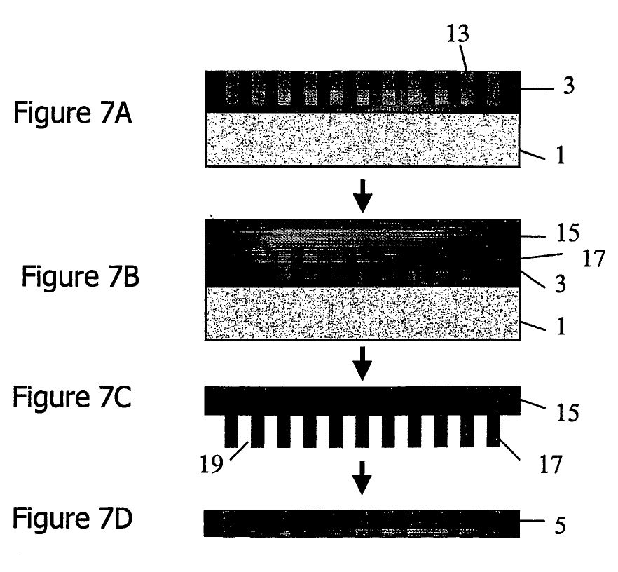

FIGS. 7A-D illustrate an alternative method of forming island using a templated nanopore array. As shown in FIG. 7A, the metal oxide nanopore array 3 on substrate 1 is formed using any suitable method described above. Then, a conformal template material 15 is deposited over the array 3, as shown in FIG. 7B. The conformal template material 15 may comprise any material which can nanopore array should not be considered limiting on the scope of the invention.

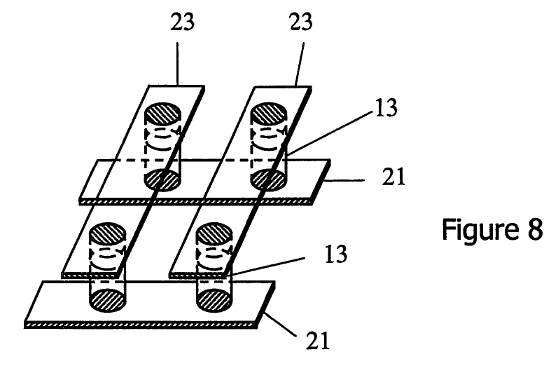

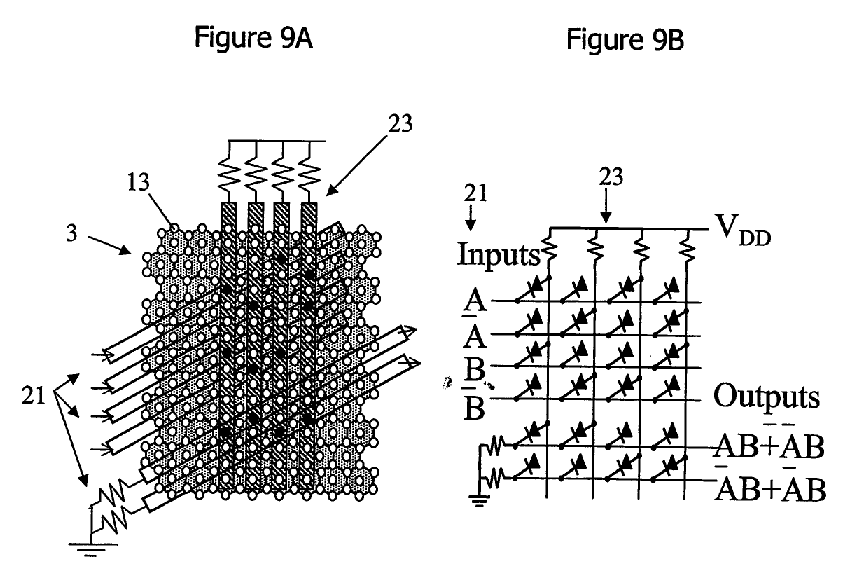

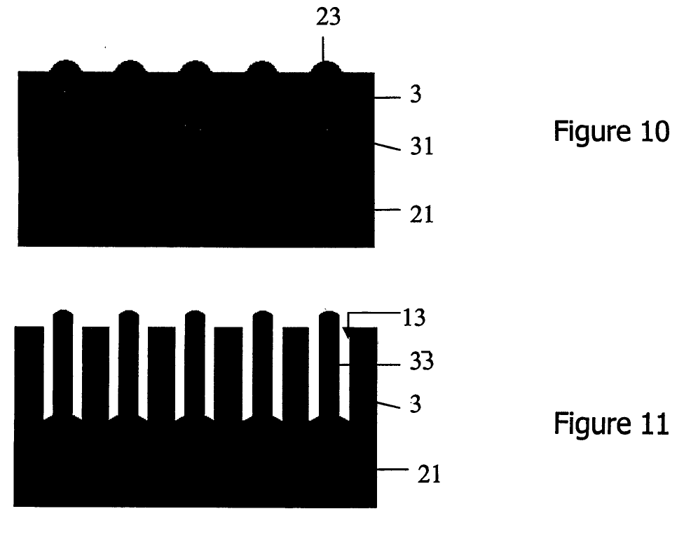

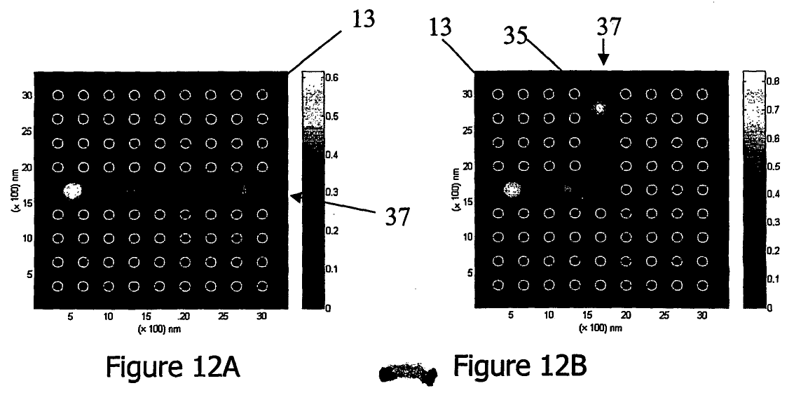

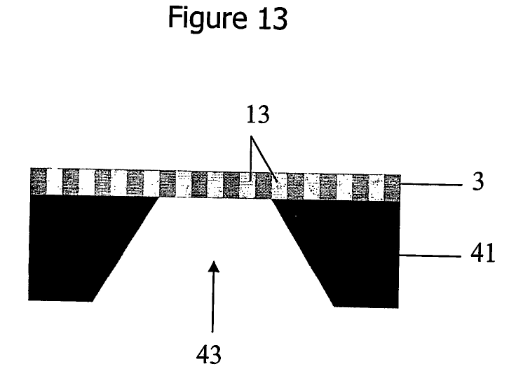

In one preferred embodiment, placing ordered arrays of nanoporous alumina onto a silicon wafer provides for several microelectronic applications. The alumina pattern may be used as a template for the later manipulation of the underlying silicon substrate. For example, the nanopores may be used to direct deep etching of the silicon substrate or wafer, as described above. Subsequently, a silicon oxide or other capacitor dielectric may be deposited into the nanowells or nanopores 13 produced by the deep etching along so as to produce a folded capacitor, as illustrated in FIG. 8. In the capacitor shown in FIG. 8, the bottom electrode 21 is located below the nanopores and the top electrode 23 is deposited over the nanopore array 3. Thus, in this embodiment, the nanopores 13 in the substrate are etched using the bottom electrode material as an etch stop. Such capacitors have a very high density across the face of the chip and could be used in a variety of applications commonly known in the microelectronics art.

If desired, access transistors, such as MOSFET, MESFET, bipolar and BiCMOS transistors, or other switching elements, such as diodes, may be fabricated in the substrate between the nanopores, above the nanopores (i.e., above the substrate), or below the nanopores (i.e., in the substrate). Alternatively, transistors or diodes may be formed in the nanopores themselves. For example, pillar type (i.e., vertical) transistors and/or diodes may be formed in the nanopores. The transistors may be fabricated before or after the formation of the nanopores. If the transistors are located above or below the nanopores, then the transistors may be fabricated in a separate substrate which is then bonded or otherwise adhered to the substrate containing the nanopores, or the transistors may be fabricated in a layer deposited above or below the nanopores. The transistors are connected to one of the electrodes 21, 23 of the capacitor to form a dynamic random access memory (DRAM).

In another preferred embodiment of the present invention, the nanopore array is used in a read only memory (ROM) device. For example, the dielectric material located in the nanopores may be used as an antifuse dielectric to form an antifuse device rather than a capacitor device. In an antifuse, the dielectric prevents current flow between the electrodes 21, 23 during reading of the device (a “0” memory state). However, when a current or voltage above a predetermined threshold voltage is provided between the electrodes 21, 23, the dielectric material is ruptured or blown, and a conductive link is formed between the electrodes 21, 23. Thereafter, the conductive link provides a current path between the electrodes 21, 23 during reading of the device (a “1” memory state).

Alternatively, a conductive fusible link between the electrodes 21, 23 may be located in the nanopores to form a fuse device. In a fuse device, the link allows current flow between the electrodes 21, 23 during reading of the device (a “1” memory state). However, when a current or voltage above a predetermined threshold voltage is provided between the electrodes 21, 23, the conductive link is ruptured or blown to sever the current path formed between the electrodes 21, 23. Thereafter, there is no current path between the electrodes 21, 23 during reading of the device (a “0” memory state). The antifuse or fuse devices may be incorporated into a field programmable gate array (FPGA), which is schematically illustrated in FIG. 9A and which is illustrated as a circuit schematic in FIG. 9B.

In alternative embodiments of the present invention, semiconductor, metal and other materials may be placed into the nanopores. For example, a light emitting diode, laser diode or other light emitting device may be formed in each nanopore, as illustrated in FIG. 10. For example, a PN junction 31 of suitable semiconductor materials formed in a nanopore acts as a light emitting diode or a laser diode, if the lasing conditions are satisfied. For example, the PN junction may comprise any two or more suitable III-V, II-VI or IV-IV layers of semiconductor material used for radiation emission upon the application of current. In this case, one or both electrodes 21, 23 is made from a radiation transparent conductive material, such as indium tin oxide. When a voltage is applied between the electrodes, the PN junction emits radiation, such as UV, IR or visible light.

Alternatively, the PN junction may be used as a photodetector or photodiode. In this case, when radiation is incident on the PN junction through a radiation transparent electrode, a photo current flow is generated between the electrodes. In should be noted that other suitable radiation emission and detecting materials or devices may be located in the nanopores instead of semiconductor PN junctions.

In another preferred embodiment, the nanopore array may be used to form ultra dense, high aspect metallization vias for a device, such as a solid state microdevice. Solid state microdevices, such as semiconductor memory and logic devices for example, contain individual devices, such as transistors, diodes and capacitors, interconnected by one or more levels of metallization or interconnects that extend through vias in one or more insulating layers. The nanopore array may be used to form high aspect vias for such metallization or interconnects.

For example, in one preferred aspect, the anodically oxidized metal oxide layer comprises the insulating layer which is located over the solid state device(s) and which contains the metallization. In this case, the nanopores are etched down to the underlying device or to a lower level of metallization to form a via. A conductive interconnect or plug, such as a metal or polysilicon interconnect or plug, is then formed in the via by any suitable method, including the plating methods described above, to contact the underlying device or metallization level.

In another preferred aspect, the nanopore array is formed over a patterned insulating layer, which is in turn located over the device(s). The nanopore array is used as a template or mask to etch the vias in the insulating layer. In other words, the etching medium is provided through the nanopores to form the vias in the insulating layer. The metal oxide layer containing the nanopores may be left in place or removed after the via etching, and a conductive interconnect or plug described above is formed in the vias.

In another preferred embodiment, a magnetic material, such as a ferromagnetic metal material, may be placed into the nanopores that are etched into the silicon and/or which are located in the metal oxide layer, and ultra-high density magnetic storage devices could be produced. Alternatively, packing magnetic materials into the nanopores may be used to produce a high sensitivity magnetic sensor. For example, a giant magnetoresistive effect device, such as a spin valve magnetoresistive device (SVMR) may be formed in the nanopore array. A SVMR device contains two ferromagnetic layers, a nonmagnetic layer between the two ferromagnetic layer and an antiferromagnetic layer located adjacent to one of the ferromagnetic layers. Any one or more of these layers may be located in the nanopores. Background for magnetic devices may be found in Routkevitch et al., IEEE Trans. Electron Dev. 43 (10): 1646 (1996); Black et al., Appl. Phy. Lett. 79:409 (2001); Metzger et al., IEEE Trans. Magn. 36 (1) :30 (2000).

Other materials located in the nanopores may comprise carbon nanotubes. For example, as illustrated in FIG. 11, the nanopore array may be used in a high resolution digital display which uses carbon nanotube electron emitters. By plating a suitable catalyst material, such as iron or magnetic cobalt at the bottom of a pore 13, such as by the selective electroplating method, and subsequently supplying a carbon nanotube source material, such as a source gas, for example ethylene gas, and heat, one or more carbon nanotubes 33 selectively form in each nanopore. The self aligned nanotube array act as an electron emitter array when an external stimulus, such as a voltage is applied to the nanotubes 33 from electrode 21. The electron emissions that the carbon nanotubes create strike an electron sensitive material, which emits radiation. Thus, the nanotube array could be used in flat-panel displays. In addition, if the substrate on which the alumina nanopores were formed is plastic, then flexible, high resolution displays could be produced. Structured nanopores could be used further as a guide or template for the ordering or stacking of not only carbon nanotubes, but of any material. Background on the use of carbon nanotubes may be found in Li et al., Appl. Phys. Lett. 75(3) :367 (1999); Bae et al., Adv. Mat. 14(4) :277 (2002); Choi et al., Appl. Phys. Lett. 79(22); 3696 (2001).

In another preferred embodiment of the present invention, the nanopore array is used in a photonic device. Placing an appropriately optically-active substance into pores (or in deeply etched holes generated using the pores as a mask) may also produce nano-machines that could be used to manipulate light. Optical fibers that are used industrially to transmit information via light require decoding and routing of that information. Currently, the routers that are employed are limited by the ability to bend the light beam while retaining all of the information contained within the beam. By packing an appropriate material into alumina nanopores and the surrounding material, an optical micro device called a photonic crystal may be produced. Photonic crystals have been shown to be highly effective in bending light beams relatively sharply, while retaining the information contained within the beam.

Alternatively, the photonic crystal may be formed as illustrated in FIGS. 12A and B. In this preferred aspect, the substrate comprises a radiation transmissive material. For example, the substrate may comprise a waveguide which comprises an optical core sandwiched between a cladding. The nanopores 3 extend through the core. Since the radiation 35 preferably travels through the uninterrupted core rather than through the nanopores, areas of the core that lack the nanopores form an optical (i.e., radiation) path 37. The arrangement of the nanopores determines the shape of the optical path. Thus a straight or curved optical path may be formed, as shown in FIGS. 12A and B, respectively. It should be noted that the nanopore array with the light path is also an ordered, single domain array with a predetermined pattern, and the light path is not a defect, since it is intentionally added to the array.

In another preferred embodiment of the present invention, the nanopore arrays are used in the production of fuel cells. Using the alumina nanopores as a mask for deep etching, a large capacity physical storage media may be created in the substrate. This media may be used to store hydrogen which is used a fuel in the fuel cell. Alternatively, the deeply-etched pores may be filled with an appropriate electrolyte material, such as polytetrafluoroethylene and high voltages between wells could be generated and, as such, high capacity fuel cells could be manufactured. Background material on fuel cells may be found in Carrette et al., Fuel Cells, 1(1) : 5-39 (2001).

In another preferred embodiment of the present invention, deep etching of a substrate using nanopore structures can also produce materials which function as a chemical catalyst. For example, titanium oxide forms nanopores after oxidation of elemental titanium. Such nanopores have extremely large surface areas making them ideal for use as catalysts, particularly since titanium oxide has catalytic properties. Background on catalytic properties of titanium oxide may be found in Gong et al., Mat. Res. 16(12): 3331 (2001); Yamashita et al., Appl. Surf. Sci. 121/122:305 (1997).

In a still further application, as illustrated in FIG. 13, ordered nanoporous membranes 41 may be obtained by introducing an additional intermediate or release layer of material between the nanoporous material and the substrate. The intermediate layer may be composed of a material that is able to be etched away using a chemical etching process. The procedure of obtaining ordered arrays of nanopores onto a substrate may proceed as described above. However, the nanopore arrays form on the surface of the intermediate layer. Following formation of the nanopores, the intermediate layer is etched away, thus detaching the nanopore array. The lower, closed portion of the pores could then be opened by chemical treatment such as etching. The resulting material would functionally operate as a very fine membrane. Such membranes have utility in a variety of chemical and biochemical separations applications. Alternatively, the release or intermediate layer is omitted, and the substrate is selectively removed such as by polishing, CMP, grinding, selective etching or other suitable methods, after forming the nanopore array. Alternatively, the nanopore array may be formed in an upper part of the substrate and thereafter at least a portion 43 of the lower part of the substrate 1 below the nanopores is selectively removed. For example, the upper and lower portions of the substrate may be formed from different materials or oppositely doped semiconductor materials, where the lower material may be selectively etched or polished away with respect to the upper material. The membranes may be antibody based nanomembranes used for enentiomeric drug separation, as well as for absorbent media and catalytic surface and supports. Background on nanoporous filters may be found in Lee et al., Science; 296:2198 (2002).

Thus, highly-ordered nanopore arrays with controlled symmetry are formed onto a foreign substrate surface. The ordered arrays of nanopores are arranged over large areas of an arbitrary substrate. Using holographic lithographic patterning of photoresist layer, a regular pattern of ridges and recesses, such as corrugations may be generated on the surface of a substrate. A material, such as aluminum, may then be deposited onto the patterned surface in a thickness such that the pattern is maintained across the surface of the material. This material should be able to form nanopore arrays. The nanopores typically form in the crevices of the recesses or corrugations. Accordingly, nanopores are arranged regularly across the entire face of the substrate. The regular arrangement of nanopores allows for the small scale manipulation of substrate. Accordingly, various nanoscale electronic, photonic, and chemical devices may be designed, engineered, and constructed.

Although the invention has been described in terms of particular embodiments in an application, one of ordinary skill in the art, in light of the teachings herein, can generate additional embodiments and modifications without departing from the spirit of, or exceeding the scope of, the claimed invention. Accordingly, it is understood that the drawings and the descriptions herein are proffered only to facilitate comprehension of the invention and should not be construed to limit the scope thereof.

1. An ordered, single domain nanopore array having a macroscale area located in a first material, wherein the first material comprises a metal oxide film formed by anodic oxidation of a metal film or a non metal oxide material in which the nanopore array is formed using a metal oxide nanopore array template.

2. The array of claim 1, wherein the first material comprises a metal oxide film formed by anodic oxidation of a metal film

3. The array of claim 1, wherein the first material comprises a semiconductor material.

4. The array of claim 1, wherein the array is substantially defect free in the single domain.

5. The array of claim 4, wherein the single domain nanopore array comprises nanopores arranged in a predetermined ordered symmetric pattern.

6. The array of claim 5, wherein the single domain nanopore array comprises nanopores arranged in an ordered square or triangular symmetric pattern.

7. The array of claim 4, wherein the single domain nanopore array comprises nanopores arranged in a one dimensional grating pattern where the nanopores are aligned in order along a grating vector direction but are not aligned along a grating line direction.

8. The array of claim 4, wherein the single domain nanopore array comprises a plurality of cells, each cell comprising nanopores arranged in a predetermined ordered symmetric pattern.

9. The array of claim 1, wherein the macroscale area comprises an area of at least one centimeter.

10. The array of claim 2, wherein the metal oxide film is located over a patterned substrate having an ordered pattern of depressions located in correspondence with an ordered pattern of nanopores in the metal oxide film.

11. The array of claim 1, wherein the nanopore diameter is 500 nm or less.

12. The array of claim 11, wherein the nanopore diameter is about 5-10 nm.

13. The array of claim 1, wherein the nanopores are filled with a second material different from the first material.

14. A device comprising a nanopore array having an ordered predetermined pattern of nanopores in a first layer of the device.

15. The device of claim 14, wherein the device comprises a photonic crystal comprising an optically transmissive layer and the nanopore array located in the optically transmissive layer, such that an optical path is formed in predetermined nanopore free areas of the optically transmissive layer that are bounded by the nanopores of the nanopore array.

16. The device of claim 14, wherein the device comprises an electronic device.

17. The device of claim 16, wherein the device comprises a memory device comprising an array of capacitors, wherein the capacitors comprise a capacitor dielectric or a capacitor ferroelectric material located in the pores of the first layer and capacitor electrodes located on either side of the first layer.

18. The device of claim 16, wherein the electronic device comprises a programmable array device comprising an array of fusible links or an antifuse dielectric located in the pores in the first layer and electrodes located on either side of the first layer.

19. The device of claim 14, wherein the device comprises a radiation emitting or detecting device comprising a radiation emitting or radiation sensitive material located in the pores of the first layer.

20. The device of claim 14, wherein the device is selected from at least one of a magnetic sensor comprising a magnetic material located in the pores in the first layer, a fuel cell storage media, a display device comprising carbon nanotubes located in the pores in the first layer, a chemical catalyst, a battery comprising electrodes located in the pores of the first material and a nanoporous membrane.

21. The device of claim 14, wherein the nanopore array comprises a single domain nanopore array containing nanopores arranged in a predetermined ordered symmetric pattern in a macroscale area and the nanopores are filled with a second material different from a material of the first layer.

22. A method of making a nanopore array with a controlled first pattern, comprising:
providing a substrate comprising a first surface having a first pattern;
depositing a first material capable of forming nanopores onto said first surface having the first pattern; and
anodically oxidizing said first material to form the nanopore array with the controlled first pattern in the anodically oxidized first material.

23. The method of claim 22, further comprising:
forming a photoresist layer on the first surface;
patterning the photoresist layer to form a patterned photoresist layer; and
etching the first surface using the photoresist layer as a mask to form the first pattern in the first surface.

24. The method of claim 23, wherein the step of patterning the photoresist layer comprises holographically exposing the photoresist layer and selectively removing portions of the photoresist layer after the exposing step to form a controlled photoresist pattern.

25. The method of claim 24, wherein the step of holographically exposing comprises holographically exposing the photoresist layer a plurality of times while rotating the substrate and the exposing beam relative to each other between exposures to form a controlled three dimensional pattern in the photoresist layer.

26. The method of claim 23, wherein the first material contains first depressions which correspond to second depressions in the first pattern in the first surface of the substrate and the nanopores are selectively formed in the first depressions.

27. The method of claim 23, wherein the first material comprises an anodically anodizable metal.

28. The method of claim 22, further comprising etching the substrate using the anodically oxidized first material as a mask to form a nanopore array in the substrate and removing the anodically oxidized first material after the step of etching the substrate.

29. The method of claim 28, further comprising filling the nanopores in the substrate with a second material to form a device.

30. The method of claim 29, wherein the second material comprises a metal interconnect which contacts a solid state device on the substrate or a lower level of a solid state device metallization.

31. The method of claim 22, further comprising filling the nanopores with a second material to form a device.

32. The method of claim 22, wherein the step of filling comprises selectively filling the nanopores with a metal by electroplating.

33. The method of claim 32, further comprises selectively vapor depositing a material on the metal located in the nanopores.

34. The method of claim 22, further comprising anodically anodizing the first material a plurality of times under different conditions to form a plurality of separated cells, each cell comprising nanopores arranged in a predetermined ordered symmetric pattern.

35. The method of claim 22, further comprising:
placing a conformal template material into the nanopores, such that the template material comprises a plurality of ridges which extend into the nanopores; and
removing the template material containing the ridges from the nanopores.

36. The method of claim 22, wherein:
the step of providing a substrate comprising a first surface having a first pattern comprises forming a first photoresist pattern on the substrate; and
the step of depositing the first material comprises depositing a metal film onto the first photoresist pattern.

37. The method of claim 22, wherein the step of providing a substrate comprising a first surface having a first pattern comprises:
forming a hardmask layer over the substrate;
forming a two dimensional photoresist pattern on the hardmask layer;
forming a hardmask by etching the hardmask layer using the photoresist pattern as a mask; and
forming the first pattern by etching the substrate using the hardmask as a mask.

38. The method of claim 22, wherein the step of providing a substrate comprising a first surface having a first pattern comprises:
forming a hardmask layer over the substrate;
forming a first one dimensional photoresist pattern having grating lines extending in a first direction on the hardmask layer;
etching the hardmask layer using the first photoresist pattern as a mask;
removing the first photoresist pattern;
forming a second one dimensional photoresist pattern having grating lines extending in a second direction different from the first direction on the hardmask layer;
forming a hardmask by etching the hardmask layer using the second photoresist pattern as a mask;
removing the second photoresist pattern; and
forming the first pattern by etching the substrate using the hardmask as a mask.

39. A method of making a nanopore arrays with a controlled pattern, comprising:
providing a metal film capable of forming nanopores;
photolithographically patterning a first surface of the metal film to form a controlled pattern of depressions in a first surface of the metal film; and
anodically oxidizing said metal film to selectively form the nanopores in the depressions in the anodically oxidized metal film.

40. The method of claim 39, further comprising:
forming a photoresist layer on the first surface of the metal film;
patterning the photoresist layer to form a patterned photoresist layer; and
etching the first surface of the metal film using the photoresist layer as a mask to form the first pattern in the first surface of the metal film.

41. The method of claim 40, wherein the step of patterning the photoresist layer comprises holographically exposing the photoresist layer and selectively removing portions of the photoresist layer after the exposing step to form a controlled photoresist pattern.
